(19) United States
(12) Patent Application Publication
(10) Pub. No.: US 2009/0281369 A1
(43) Pub. Date: Nov. 12, 2009

(54) Urethral Spring, Implantation Tools, and Method of Treating Incontinence

(75) Inventor: Randy L. Morningstar, Brooklyn Park, MN (US)

Correspondence Address: COLOPLAST A/S;Attention: Corporate Patents, Holtedam 1, DK-3050 Humlebaek, DK

(73) Assignee: Coloplast A/S, Humlebaek (DK)

(21) Appl. No.: 12/435,411

(22) Filed: May 5, 2009

(30) Foreign Application Priority Data
May 9, 2008 (DK) PA 2008 00662

(51) Int. Cl. A61F 2/04

(52) U.S. Cl. 600/30

(57) ABSTRACT

A urethral spring comprises a spring body having a selected spring force, a first end, and a second end. The urethral spring further comprises a first urethra contacting portion at the first end of the spring body, and a second urethra contacting portion at the second end of the spring body. When the urethral spring is at rest corresponding to urethral coaptation, the spring body generally has an at rest shape. When the urethral spring is flexed corresponding to intentional voiding, the spring body generally has a flexed shape.

### FIELD OF THE INVENTION

The present invention relates generally to implantable devices for treatment of urinary incontinence. The invention relates specifically to a urethral spring for treating urinary incontinence, urethral spring implantation tools for implanting the urethral spring, and a method of treating incontinence using the urethral spring with the urethral spring implantation tools.

### BACKGROUND OF THE INVENTION

Urinary incontinence is, generally, a well-known medical condition that can affect patients regardless of gender. Urinary incontinence may occur due to a variety of factors including, for example, physical injury or sedation, pelvic organ surgeries such as hysterectomies in females or prostatectomies in males, and, simply, anatomical degradation due to advanced age. Typically, a healthy and physically effective urethra is flat or “reed” shaped, exhibiting what is known in the art of urologic medicine as “coaptation”. The urethra normally functions in response to muscular or other anatomical actions to modify its shape, i.e., to diminishing its coaptation during intentional voiding, thereby controlling passage of urine therethrough. But a urethra of an incontinent patient is often not capable of maintaining, or even attaining, a normal reed shape.

As known to those of skill in the art of urologic medicine, methods of treatment of urinary incontinence and minimization of its undesirable effects have included, for example, surgical implantation of suburethral slings, introduction of bulking agents, implantation of urethral constriction devices, and, simply, use of absorbent garments. As known to those in the urologic arts, a suburethral sling acts to lift the urethra, thereby flattening and repositioning it to achieve the aforementioned coapted reed-like shape. A bulking agent acts to add volume or “bulk” to urethral tissue, thereby tending to coapt or close off the urethra so that leakage of urine therethrough may be minimized. Urethral constriction devices are typically electro-mechanical or hydraulically actuated, and act to coapt or close off the urethra by squeezing it shut. Finally, absorbent garments do not act on the urethra, but simply absorb urine that leaks out in an incontinence episode.

These known responses to incontinence have suffered from several drawbacks. For example, treatments requiring invasive surgical procedures can lead to undesirable side effects or outcomes such as infection, erosion of the urethra or surrounding anatomical tissue, and continued incontinence because of less than optimal device placement or tensioning. Slings, in particular, often utilize mesh type materials that, when implanted, may have little or no adjustability and therefore can be problematic or ineffective. Use of absorbent garments such as adult diapers can inhibit patient mobility and activities and can also be unattractive and psychologically undesirable.

Thus, there exists a need for urethral springs for treatment of male and female incontinence that may overcome deficiencies of known incontinence treatments. In particular, such urethral springs may provide desired urethral coaptation. Also, such urethral springs may be easier to surgically implant, less invasive, and suitable for out-patient or office procedures, when compared to known incontinence treatments.

### SUMMARY OF THE INVENTION

In accordance with basic aspects of the present invention, a urethral spring comprises a spring body having a selected spring force, a first end, and a second end. The urethral spring further comprises a first urethra contacting portion at the first end of the spring body, and a second urethra contacting portion at the second end of the spring body. When the urethral spring is at rest corresponding to urethral coaptation, the spring body generally has an at rest shape. When the urethral spring is flexed corresponding to intentional voiding, the spring body generally has a flexed shape.

Also in accordance with basic aspects of the present invention, a method of manufacturing a urethral spring comprises steps of providing a spring material having a selected spring force and forming a spring body from the selected spring material. The spring body is formed by defining a first end, a second end, a first urethra contacting portion at the first end, and a second urethra contacting portion at the second end.

Additionally in accordance with basic aspects of the present invention, urethral spring implantation tools for implanting the urethral spring comprise a barrel having a urethra contacting end and an opposite insertion end. The barrel has a size that is capable of housing a urethral spring therewithin. The tools further comprise a spring advancing device having a urethral spring attachment end and an opposite insertion end. The spring advancing device has a size that is capable of movement within the barrel and is capable of attachment to, and detachment from, the urethral spring.

Further in accordance with basic aspects of the present invention, a method of use of the urethral spring to treat incontinence comprises steps of (a) surgically exposing a urethra to be treated, the urethra having a first portion and a second portion, (b) providing a urethral spring, having a spring body of a selected spring force and including a first end, a second end, a first urethra contacting portion at the first end, and a second urethra contacting portion at the second end, (c) providing urethral spring implantation tools, including a barrel having a urethra contacting end and an opposite insertion end, and a spring advancing device having a urethral spring attachment end and an opposite insertion end, (d) placing the urethral spring in the barrel of the urethral spring implantation tools, (e) attaching the spring attachment end of the spring advancing device to the urethral spring, (f) placing the urethra contacting end of the barrel against the urethra, (g) urging the insertion end of the spring advancing device, while attached to the urethral spring, toward the urethra contacting end of the barrel and the urethra, such that the first urethra contacting portion of the urethral spring is implanted into the first portion of the urethra and the second urethra contacting portion of the urethral spring is implanted into the second portion of the urethra, (h) detaching the spring advancing device from the urethral spring, and (i) removing the spring implantation tools.

### BRIEF DESCRIPTION OF THE DRAWINGS

FIG. 1 is an illustration of an example of a urethral spring of the present invention.

FIG. 2 is a perspective illustration of a portion of a normal, continent urethra in a condition of coaptation.

FIG. 2a is a perspective illustration of the normal, continent urethra of FIG. 2 in a condition of intentional voiding.

FIG. 2b is a perspective illustration of a portion of an abnormal, incontinent urethra in a condition of inadequate coaptation.

FIG. 3 is a perspective illustration of the urethra of FIG. 2b, including a urethral spring of the present invention for coaptation of the urethra.

FIG. 3a is a front view of the urethra and urethral spring shown in FIG. 3.

FIG. 4 is a perspective illustration of a component of urethral spring implantation tools of the present invention.

FIG. 4a is an illustration of the component of FIG. 4, with a urethral spring therewithin, ready for implantation surgery, and with another component of urethral spring implantation tools of the present invention.

FIG. 4b is a perspective illustration of the urethral spring implantation tools of FIGS. 4 and 4a, with the urethral spring being deployed therefrom in implantation surgery.

### DETAILED DESCRIPTION OF THE INVENTION

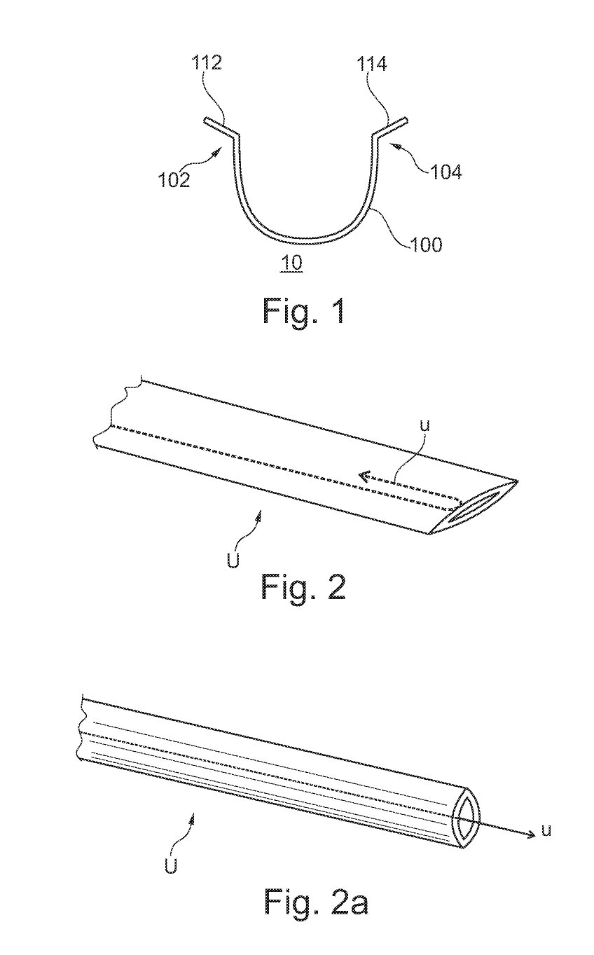

Illustrated in FIG. 1 is an example of a urethral spring 10 of the present invention. In this example, urethral spring 10 could include a spring body 100 having a selected spring force or spring constant K, a first end 102, and a second end 104. First end 102 could include a first urethra contacting portion 112, and second end 104 could include a second urethra contacting portion 114. When urethral spring 10 is at rest, corresponding to urethral coaptation as will be further described relative to a method of surgical implantation of spring 10, spring body 100 generally has an at rest shape. As shown in the example of FIG. 1, this at-rest shape is generally “U” shaped. When urethral spring 10 is flexed, corresponding to intentional voiding as will be further described, spring body 100 generally has a flexed shape (not illustrated in FIG. 1). The selected spring material could be preferably chosen so that (i) the spring force or spring constant K of spring 10 would be sufficient to provide urethral coaptation when urethral spring 10 is implanted in a patient's urethra, and (ii) it may be exceeded as desired during intentional voiding, as will also be further described. In a typical embodiment of spring 10, constant K could be selected from a range of 20 grams force to 90 grams force.

Although not illustrated, a method of manufacturing urethral spring 10 could be initiated by providing a selected spring material having a selected spring force or spring constant K. As aforementioned, the selected spring material could be preferably chosen so that the constant K would be in a range of 20 grams force to 90 grams force that may be sufficient to provide for urethral coaptation and also allow for intentional voiding. The spring material could preferably be of a metallic wire-like, and biomaterial stock such as “Elgiloy SST” or “Nitinol”. As described above relative to an example of spring 10 shown in FIG. 1, the spring material could then be formed into spring body 100 having first end 102 and first urethra contacting portion 112, and second end 104 having second urethra contacting portion 114.

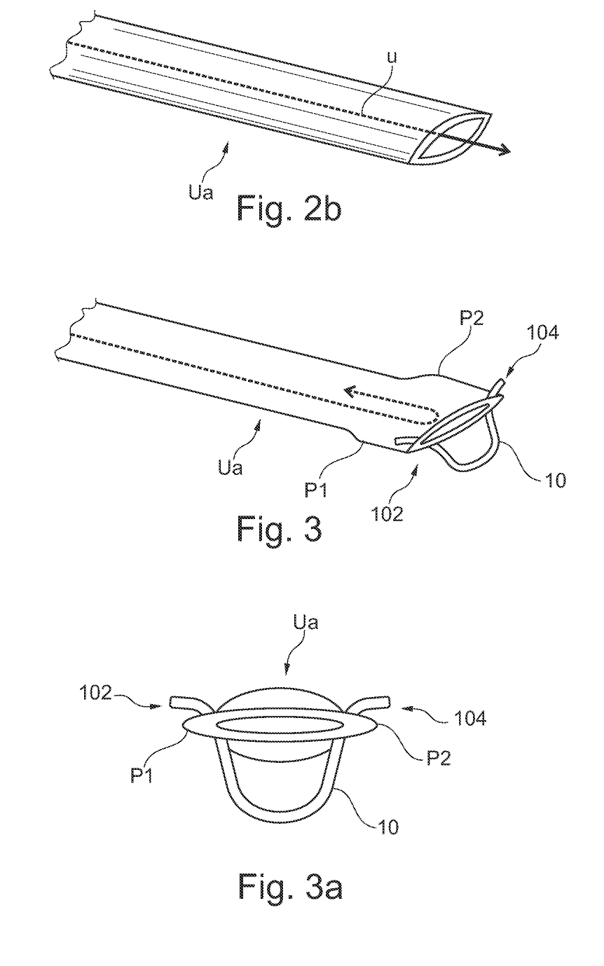

For reference, illustrated in FIGS. 2 and 2a are examples of portions of a normal, continent urethra U in conditions of coaptation and intentional voiding, respectively. Urine u is shown in FIG. 2 as being inhibited from flowing through the coapted urethra U, while a stream of urine u is shown as flowing through the urethra U in FIG. 2a during intentional voiding. In contrast, FIG. 2b illustrates an example of an abnormal, incontinent urethra Ua in a condition of inadequate coaptation such that urine u undesirably leaks through the abnormal, incontinent urethra Ua.

Illustrated in FIGS. 3 and 3a is the abnormal, incontinent urethra of FIG. 2b, with a urethral spring 10 surgically implanted therein to provide more normal coaptation as will be described below relative to a method of surgical implantation of spring 10.

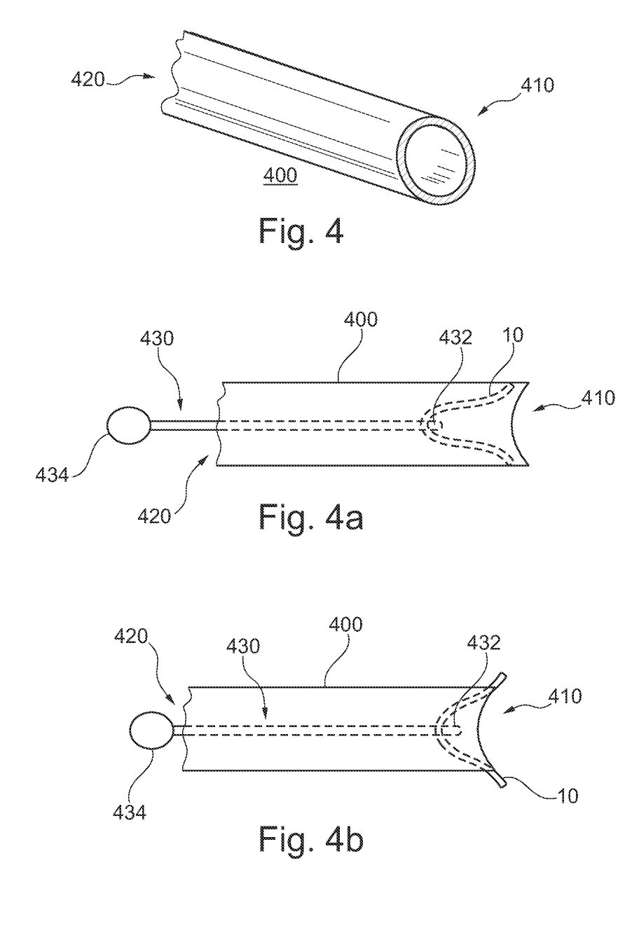

Illustrated in FIGS. 4, 4a, and 4b are examples of urethral spring implantation tools of the present invention. In FIG. 4, a barrel 400 is shown as an example of a component of urethral spring implantation tools. Barrel 400 could include a urethra contacting end 410 and an opposite insertion end 420. Barrel 400 could preferably be cylindrical, and have a size that is capable of housing urethral spring 10 therewithin as will be described. In FIG. 4a, spring 10 is depicted as being loaded within barrel 400 and ready for implantation surgery. Also shown in the drawing is an example of a spring advancing device 430. Device 430 could have a urethral spring attachment end 432 and an opposite insertion end 434. Device 430 could preferably have a size that is capable of movement within barrel 400. Device 430 could also be preferably capable of attachment to, and detachment from, urethral spring 10 at urethral spring attachment end 432 as will be described relative to a method of surgical implantation of spring 10.

FIG. 4b is a perspective illustration of the urethral spring implantation tools of FIGS. 4 and 4a, with the urethral spring being deployed therefrom in implantation surgery. In FIG. 4b, spring 10 is depicted as being urged out of or deployed from barrel 400 during implantation surgery, by way of spring advancing device 430.

With reference now to all of the drawings, a method of treating urinary incontinence may be initiated by surgically exposing an abnormal, incontinent urethra to be treated (e.g., the abnormal, incontinent urethra Ua shown in FIGS. 3-3a). Subject urethra Ua may be defined by a first portion P1 and a second portion P2. A urethral spring 10 may be provided, having a spring body 100 of a selected spring force or spring constant K and including a first end 102 having a first urethra contacting portion 112, and a second end 104 having a second urethra contacting portion 114. Urethral spring implantation tools may also be provided, including a barrel 400 having a urethra contacting end 410 and an opposite insertion end 420, and a spring advancing device 430 having a urethral spring attachment end 432 and an opposite insertion end 434. Urethral spring 10 may then be placed in barrel 400 of the urethral spring implantation tools, and spring attachment end 432 of device 430 may be attached to spring 10 within barrel 400 as shown in FIG. 4a. Urethra contacting end 410 of barrel 400 may then be placed against the urethra Ua, whereupon insertion end 434 of device 430 may be urged toward urethra contacting end 410 of barrel 400 and toward urethra Ua. In such manner, first and second urethra contacting portions 112 and 114 of spring 10 may be implanted, respectively, into the first and second portions P1 and P2 of the urethra Ua as particularly shown in FIGS. 3-3a. Spring advancing device 430 may then be detached from spring 10, and the spring implantation tools may be removed leaving spring 10 in urethra Ua as desired. The exposed urethra may then be surgically restored to its pre-operative state, with spring 10 in place, by any suitable technique.

It is to be particularly appreciated and understood by those of skill in the medical art of urology that the spring of the present invention is designed to respond to increases and decreases in intra-bladder pressure, corresponding to filling and emptying of urine therein, respectively. When the bladder is for the most part empty, pressure within the bladder and the urethra is relatively low and thus an inherent spring force or constant of the spring material may act to close the urethra. Conversely, as the bladder fills and pressure therein and in the urethra is increased, the inherent spring force or constant of the spring material may be exceeded which causes it to flex and thereby allow the urethra to open so that normal, volitional voiding can occur.

The urethral spring of the present invention may be implanted in so-called “minimally invasive” surgery, via a transvaginal approach in female patients or a perineal approach in male patients. The spring of the present invention may also be capable of being simply removed in minimally invasive surgery, with only a small incision. To accomplish these objectives, this novel and heretofore unknown urethral spring could be placed through an outer lumen of a subject urethra as aforedescribed to pull it into a “reed” shape resulting in urethral coaptation. As may be necessary or desired, several springs could be placed along the length of the urethra to provide continence. The spring of the present invention may leave the urethra and surrounding tissue “as-is”, but it essentially may cause the same desired effect as a urethral sling.

The urethral spring of the present invention is simple and could be alternatively manufactured with several selected spring forces to accommodate desired urethral closure tension and coaptation with regard to a particular patient's urethra.

It is to be understood that collectively throughout this description, the term “urethral spring” has been characterized as a surgically implanted device for providing desired urethral coaptation. However, this term is not intended to be limited to urethral applications but could also include other implantable devices involving desired restriction of a vessel. Thus, novel aspects of the present invention regarding implantable devices per se will be appreciated by those in the surgical arts to be capable of use in, and beneficial to, virtually any implantable devices—even those outside of the field of urology.

It is to be appreciated from the foregoing disclosure that the present invention uniquely and advantageously satisfies a long-felt need for a urethral spring, implantation tools for implanting the urethral spring, and a method of treating incontinence using the urethral spring with the urethral spring implantation tools—which all may be utilized in minimally invasive surgery.

While the present invention has been particularly shown and described with reference to the accompanying specification and drawings, it will be understood however that other modifications thereto are of course possible; and all of which are intended to be within the true spirit and scope of the present invention. It should be appreciated that (i) components, dimensions, shapes, and other particulars of example embodiments of the invention aforedescribed may be substituted for others that are suitable for achieving desired results, (ii) various additions or deletions may be made thereto, and (iii) features of the foregoing examples may also be made in combinations thereof. It is also to be understood in general that any suitable alternatives may be employed to provide the urethral spring, implantation tools, and method of the present invention.

Lastly, of course, the choice of compositions, sizes, and strengths of various aforementioned elements of the present invention are all a matter of design choice depending upon intended uses thereof.

Accordingly, these and other various changes or modifications in form and detail of the present invention may also be made therein, again without departing from the true spirit and scope of the invention as defined by the appended claims.

What is claimed is:

1. A urethral spring, comprising:
a spring body having (i) a selected spring force, (ii) a first end, and (iii) a second end;
a first urethra contacting portion at said first end of said spring body; and
a second urethra contacting portion at said second end of said spring body,
wherein when said urethral spring is at rest corresponding to urethral coaptation, said spring body generally has an at rest shape, and wherein when said urethral spring is flexed corresponding to intentional voiding, said spring body generally has a flexed shape.

2. The urethral spring of claim 1, wherein said selected spring force is sufficient to provide urethral coaptation when said urethral spring is implanted in the urethra, and wherein said selected spring force is exceeded during intentional voiding.

3. The urethral spring of claim 2, wherein said selected spring force is in a range of 20 grams force to 90 grams force.

4. A method of manufacturing a urethral spring, comprising steps of:
providing a spring material having a selected spring force; and
forming a spring body from said selected spring material, said spring body having a first end, a second end, a first urethra contacting portion at said first end, and a second urethra contacting portion at said second end.

5. The method of manufacturing a urethral spring of claim 4, wherein said selected spring force is sufficient to provide urethral coaptation when said urethral spring is implanted in the urethra, and wherein said selected spring force is exceeded during intentional voiding.

6. The method of manufacturing a urethral spring of claim 5, wherein said selected spring force is in a range of 20 grams force to 90 grams force.

7. The method of manufacturing a urethral spring of claim 4, wherein said spring material is selected from a group consisting of “Elgiloy SST” and “Nitinol”.

8. Urethral spring implantation tools for implanting a urethral spring in a urethra, said urethral spring implantation tools comprising:
a barrel having a urethra contacting end and an opposite insertion end, said barrel having a size that is capable of housing a urethral spring therewithin; and
a spring advancing device having a urethral spring attachment end and an opposite insertion end, said spring advancing device (i) having a size that is capable of movement within said barrel and (ii) being capable of attachment to, and detachment from, said urethral spring.

9. A method of treating urinary incontinence, comprising steps of:
surgically exposing a urethra to be treated, the urethra having a first portion and a second portion;
providing a urethral spring, having a spring body of a selected spring force and including a first end, a second end, a first urethra contacting portion at said first end, and a second urethra contacting portion at said second end;
providing urethral spring implantation tools, including a barrel having a urethra contacting end and an opposite insertion end, and a spring advancing device having a urethral spring attachment end and an opposite insertion end;
placing said urethral spring in said barrel of said urethral spring implantation tools;
attaching said spring attachment end of said spring advancing device to said urethral spring;
placing said urethra contacting end of said barrel against the urethra;
urging said insertion end of said spring advancing device, while attached to said urethral spring, toward said urethra contacting end of said barrel and said urethra, such that (i) said first urethra contacting portion of said urethral spring is implanted into the first portion of the urethra and (ii) said second urethra contacting portion of said urethral spring is implanted into the second portion of the urethra;
detaching said spring advancing device from said urethral spring; and
removing said spring implantation tools.
